(19) United States
(12) Patent Application Publication
(10) Pub. No.: US 2010/0298600 A1
(43) Pub. Date: Nov. 25, 2010

(54) NANODIAMOND COMPOUNDS SYNTHESIZED BY SURFACE FUNCTIONALIZATION

(75) Inventor: Min Yung Lee, Seoul (KR)

Correspondence Address: SUGHRUE MION, PLLC, 2100 PENNSYLVANIA AVENUE, N.W., SUITE 800, WASHINGTON, DC, 20037, US

(73) Assignee: NANODIAMOND INC., Seoul (KR)

(21) Appl. No.: 12/681,808

(22) PCT Filed: Oct. 9, 2008
(86) PCT No.: PCT/KR2008/005920

§ 371 Date: Aug. 3, 2010

(30) Foreign Application Priority Data
Oct. 13, 2007 (KR) 10-2007-0103292

(51) Int. Cl. C07C 51/16; C07C 29/32; C07C 209/14; C07C 231/02

(52) U.S. Cl. 562/523; 568/876; 564/478; 564/138; 977/788

(57) ABSTRACT

Disclosed herein is a method for chemically attaching carboxyl, alcohol, amine or amide groups to the surface of nanodiamond (ND) in a liquid phase. Also disclosed herein are a functional ND compound obtained by the method and use thereof. The method includes treating synthetic ND with a size of 1 nm-1OO nm with sonication and a strong acid to provide ND-(COOH)ₙ. The ND-(COOH)ₙ compound is used as a starting material to provide ND compounds having alcohol, amine or amide groups attached to the surfaces thereof. The surface-functionalized ND compounds are characterized by using an X-ray diffractometer, FTIR, AFM, particle size analyzer and zeta sizer. The ND compounds show functionalities as well as high solubility to provide stable ND solutions in a liquid phase. Therefore, the ND compounds may be used as diamond coating agents. The powder of the ND compounds may be used as materials for producing composites of polymers, plastics, synthetic fibers, ceramics, etc., or as additives for toothpaste, shampoos, soap and cosmetic compositions.

### TECHNICAL FIELD

Disclosed herein is diamond nanoparticle, nanodiamond (ND). More particularly, disclosed herein are chemical surface functionalization technology of ND in a liquid phase and a functional diamond compound obtained thereby.

### Background Art

While diamond has been known as the most valuable jewel, it also has been recognized as a material having excellent characteristics in substantially all industrial fields including the electronic industry and chemical industry. Diamond shows many advantages including high hardness, light transmission over a wide range of wavelengths, superior chemical stability, high thermal conductivity, low heat expansion, good electrical insulating property, good biocompatibility, etc. Recently, as nanotechnology has developed markedly, methods for producing powder or thin films of diamond have been studied to accomplish effective application of such advantageous characteristics of diamond. Micro-scaled diamond powder has been already utilized in a wide spectrum of industrial fields.

Disclosed herein are diamond nanoparticles (nanodiamond, ND) having a size of 1 nm-100 nm, and a method for producing the ND. Particular examples of the process for producing ND known to date include high-temperature high-pressure processes, diamond synthesis using shock waves, chemical vapor deposition processes, detonation processes, or the like.

Particularly, ND particles having a size of 10 nm or less are designated as ultra-nanocrystalline diamond (UNCD). UNCD is ultrafine diamond crystal having a relatively uniform particle size distribution of a particle diameter of around 5 nm, and is synthesized mainly by explosive detonation. ND with a size of 10 nm-100 nm is obtained by grinding micro-scaled diamond powder synthesized by using shock waves or by a high-temperature high-pressure process mechanically and finely. In general, natural diamond is known to exhibit hydrophobicity (or oleophilicity). On the contrary, ND having a large surface area to volume ratio exhibits hydrophilicity.

ND has a crystal structure in which the core comes with a sp₃-hybridized orbital function and the surface comes with sp orbital. Therefore, the core maintains the many atoms or molecules are chemically bound to the dangling bonds. Herein, the composition of such atoms or molecules depends on the particular method by which the diamond is synthesized. Although such chemical bonds present on the diamond particles contribute to surface stabilization of the diamond particles, various functional groups may be attached to the surface of ND via new chemical reactions. In general, a higher ratio of sp₂/sp₃ provides higher reactivity of ND. For example, when ND has a particle size of 4.2 nm, the ratio even reaches 15%.

Diamond powder has been utilized as coating agents for metal surfaces, polymer and rubber composites, abrasives, oil additives, etc. Theoretically, diamond powder is colorless and transparent. Thus, when diamond powder is used as a coating agent or is dispersed into a polymer plastic material, its presence is not detected apparently. ND core is in a crystalline form, but impurities may be present around the surface of ND due to its strong surface reactivity. To remove such surface impurities of ND and to improve the applicability of ND, a surface oxidation process has been developed. However, ND is present in solution as aggregates having different sizes due to the strong interaction between ND particles and oxygen moieties with strong reactivity. As possible mechanisms for aggregation of ND, there have been suggested “soft aggregation” generated by physical adsorption among ND particles, and “hard aggregation” formed by chemical bonding among ND particles.

Surface treatment of ND may minimize aggregation of ND upon dispersing in a liquid phase so that ND exists in a single particle state. Particular examples of the known methods of such surface treatment include heat treatment of diamond powder in a vapor phase in the presence of a mixed gas of hydrogen with chlorine, or cold plasma treatment using fluorine gas. The vapor-phase surface functionalization of ND requires expensive equipments and complicated processing steps, and thus is not applicable to mass production. There have been no reported methods of attaching various functional groups to the surface of ND via a chemical process in a liquid phase. Functional ND compounds, whose surfaces have alcohol, amine, amide or other groups attached thereto via a chemical process in a liquid phase, are disclosed herein for the first time. The surface-functionalized ND compound as disclosed herein shows a high dispersibility of up to 15% in a liquid phase on the weight basis, and maintains its stable state as single particles without aggregation for a long time.

The surface-functionalized ND compound is expected to have various uses. Particularly, the ND compound may be used as a material for coating agents and lubricant oil as it is, and may be added to polymer plastics, ceramic composites, fibers, paper, toothpaste, shampoo, soap, cosmetics, etc. to impart certain functionalities thereto. Additionally, the surface-functionalized ND compound may be used as a starting material for preparing nanobiomaterial-based medicines.

### DISCLOSURE OF INVENTION

### Technical Problem

Provided is a method for preparing a surface-functionalized nanodiamond (ND) compound.

Also provided is a surface-functionalized ND compound obtained by the above-mentioned method and having a size of 1 nm-100 nm.

Also provided is a highly dispersible surface-functionalized ND compound for use in polymers, plastics, fibers, functional beverage, toothpaste, soap, shampoo, cosmetics, medicines or the like.

### Technical Solution

In an aspect, there is provided a method for surface functionalization of nanodiamond (ND) powder, which includes dispersing the ND powder in a liquid phase at a high concentration, and treating the dispersion containing ND powder dispersed therein with a strong acid. The ND powder may be dispersed in a liquid phase at a high concentration by any one process selected from the group consisting of wet milling using microbeads, sonication and a combination thereof.

In another aspect, there is provided an ND compound having COOH groups attached to the surface thereof and obtained from the above-mentioned method.

In still another aspect, there is provided a method for surface functionalization of ND powder, which includes dispersing an ND compound having COOH groups attached to the surface thereof into tetrahydrofuran (THF), and adding lithium aluminum hydride (LiAlH₄) to the resultant dispersion.

In still another aspect, there is provided an ND compound having CH₂OH groups attached to the surface thereof and obtained from the above-mentioned method.

In still another aspect, there is provided a method for surface functionalization of ND powder, which includes dispersing an ND compound having CH₂OH groups attached to the surface thereof into THF, and adding diethyl azodicarboxylate as a coupling agent and phthalimide to the resultant dispersion.

In still another aspect, there is provided an ND compound having CH₂NH₂ groups attached to the surface thereof and obtained from the above-mentioned method.

In still another aspect, there is provided a method for surface functionalization of ND powder, which includes dispersing an ND compound having COOH groups attached to the surface thereof into ethylenediamine, and adding N-[dimethylamino]-1H-1,2,3-triazo[4,5,6]pyridinylmethylene]-N-methylmethanaminium hexafluorophosphate N-oxide (HATU) to the resultant dispersion.

In still another aspect, there is provided an ND compound having CONHCH₂CH₂NH₂ groups attached to the surface thereof and obtained from the above-mentioned method.

In still another aspect, there is provided a coating agent including a surface-functionalized ND compound having a particle diameter of 1 nm-100 nm.

In still another aspect, there is provided a polymeric film including a surface-functionalized ND compound having a particle diameter of 1 nm-100 nm.

In still another aspect, there is provided plastic including a surface-functionalized ND compound having a particle diameter of 1 nm-100 nm.

In still another aspect, there is provided rubber including a surface-functionalized ND compound having a particle diameter of 1 nm-100 nm.

In still another aspect, there is provided leather including a surface-functionalized ND compound having a particle diameter of 1 nm-100 nm.

In still another aspect, there is provided a fiber including a surface-functionalized ND compound having a particle diameter of 1 nm-100 nm.

In still another aspect, there is provided paper including a surface-functionalized ND compound having a particle diameter of 1 nm-100 nm.

In still another aspect, there is provided glass including a surface-functionalized ND compound having a particle diameter of 1 nm-100 nm.

In still another aspect, there is provided ceramic including a surface-functionalized ND compound having a particle diameter of 1 nm-100 nm.

In still another aspect, there is provided a cosmetic composition including a surface-functionalized ND compound having a particle diameter of 1 nm-100 nm.

In still another aspect, there is provided toothpaste including a surface-functionalized ND compound having a particle diameter of 1 nm-100 nm.

In still another aspect, there is provided soap including a surface-functionalized ND compound having a particle diameter of 1 nm-100 nm.

In still another aspect, there is provided a shampoo including a surface-functionalized ND compound having a particle diameter of 1 nm-100 nm.

### ADVANTAGEOUS EFFECTS

Functional nanodiamond (ND) compounds designated by ND-Rₙ are obtained by the methods disclosed herein. More particularly, ND compounds represented by the formula of ND-Rₙ, wherein R is an alcohol, amine or amide group, are provided in an aqueous phase.

The functional ND compound as disclosed herein is capable of being dispersed in a solution at a high concentration. Thus, various functional groups may be attached to the surface of ND having an average of 1 nm-100 nm to functionalize the ND. Additionally, the functional ND compound shows an increased solubility in an aqueous solution as compared to existing ND powder by several tens of times, and provides a stable ND solution in the pH range from 2 to 12. Further, the functional ND compound may be applied to a polymer composite material, plastic, ceramic, fiber, toothpaste, shampoo, soap, cosmetics, or the like. In addition to the above, the functional ND compound may be utilized as a material for a medicine, as long as the pharmacological effect and stability of the functional ND are demonstrated.

### BRIEF DESCRIPTION OF DRAWINGS

Description will now be made in detail with reference to certain example embodiments illustrated in the accompanying drawings which are given hereinbelow by way of illustration only, and thus are not limitative of the methods and nanodiamond (ND) compounds disclosed herein, and wherein:

FIG. 1 is a schematic view showing a functional ND compound synthesized via a surface chemical reaction;

FIG. 2a and FIG. 2b are X-ray diffraction spectra of ND compounds represented by the formulae ND₅-(COOH)ₙ and ND₆₀-(COOH)ₙ, respectively;

FIG. 3 is a Fourier transform infrared (FTIR) spectrum of the ND₅ nanodiamond compound;

FIG. 4 is an FTIR spectrum of the ND₆₀ nanodiamond compound;

FIGS. 5a and 5b are photographic views taken by atomic force microscopy and size distribution diagrams of ND₅-(CH₂OH)ₙ and ND₅-(CH₂NH₂)ₙ, respectively;

FIGS. 6a and 6b are photographic views taken by atomic force microscopy and size distributions of ND₆₀-(CH₂OH)ₙ and ND₆₀-(CH₂NH₂)ₙ, respectively;

FIG. 7 is a size distribution diagram of the ND₅ nanodiamond compound obtained by using a dynamic light scattering particle size analyzer;

FIG. 8 is a size distribution diagram of the ND₆₀ nanodiamond compound obtained by using a dynamic light scattering particle size analyzer;

FIG. 9 is a graph showing the zeta potential measurements of the ND₅ nanodiamond compound; and

FIG. 10 is a graph showing the zeta potential measurements of the ND₆₀ nanodiamond compound.

### BEST MODE FOR CARRYING OUT THE INVENTION

Hereinafter, reference will now be made in detail to various embodiments of the methods and nanodiamond (ND) compounds disclosed herein, examples of which are illustrated in the accompanying drawings and described below. While the methods and ND compounds will be described in conjunction with example embodiments, it will be understood that the present description is not intended to limit the methods and ND compounds disclosed herein to those example embodiments. On the contrary, the methods and ND compounds disclosed herein are intended to cover not only the example embodiments, but also various alternatives, modifications, equivalents and other embodiments, which may be included within the spirit and scope as defined by the amended claims.

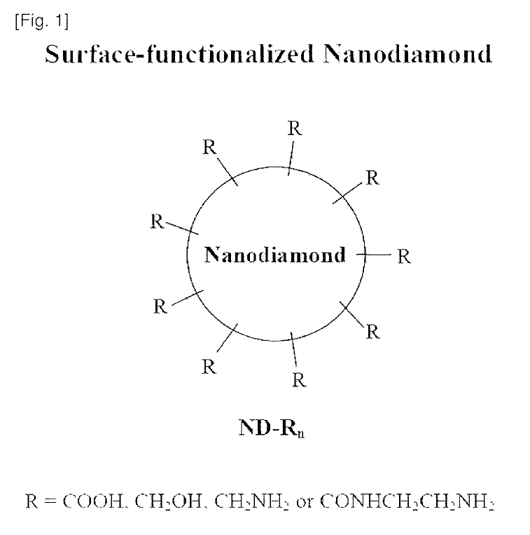

FIG. 1 is a schematic view showing the surface-functionalized ND compound as disclosed herein.

As used herein, the formula ND-Rₙ represents the surface-functionalized ND compound obtained by the method as disclosed herein. Herein, ND means nanodiamond forming the core of the compound, R represents a chemical functional group, and n represents the number of functional groups attached to the surface of ND. When the nanodiamond requires identification of its size, it is represented by the formula of NDₓ-Rₙ, wherein X means the average particle size of core ND particles but merely represents the approximate particle size.

The following two types of ND particles are used as starting materials to perform surface functionalization via the methods as disclosed herein: one is nanodiamond (ND₅) having a diameter of about 5 nm and obtained by detonation, and the other is nanodiamond (ND₆₀) having a diameter of about 60 nm and obtained by finely grinding microdiamond. Non-crystalline carbon compounds still remain on the surfaces of such ND particles, or the ND particles are surrounded by oxygen or hydrogen compounds. Further, in many cases, the ND particles form aggregates. When the ND particles are agitated in a solution of strong acid for several hours while carrying out sonication in an aqueous phase, such impurities are removed from the ND and COOH groups are formed so that the ND is dispersed in the liquid phase in a single particle state. As used herein, the formula NDₓ-(COOH)ₙ represents a surface-functionalized ND compound obtained via the above-mentioned surface treatment process.

The ND compounds represented by the formulae ND₅-(COOH)ₙ and ND₆₀-(COOH)ₙ are subjected to chemical reactions according to the methods as disclosed herein to provide functional ND compounds having alcohol, amine or amide groups attached to the surfaces thereof. The crystal structures of the ND compounds are determined by X-ray diffraction analysis. Additionally, FTIR determines whether the functional groups are attached to the surfaces of ND or not. Further, the particle sizes of the ND compounds are measured by using an atomic force microscope when they are in the form of powder, and by using a dynamic light scattering particle size analyzer when they are dispersed in a liquid phase. In addition to the above analytical methods, zeta potential measurement is used to determine the surface charges of the ND compounds.

Since the ND compounds have a high solubility in an aqueous solution or organic solvent, they may be applied to various industrial fields. Various functional groups of other polymers may be attached to the diamond compounds. Otherwise, biomolecules including nucleotides and peptides may be bound to the surfaces of the ND compounds.

### MODE FOR THE INVENTION

The following examples illustrate the methods and nanodiamond (ND) compounds disclosed herein, but are not intended to limit the same.

### Example 1

ND₅ nanodiamond powder is added to a strong acid solution containing HNO₃ (70%) and H₂SO₄ (98%) in a mixing ratio of 1:3 to introduce carboxyl groups to the surface of the ND. Next, the resultant solution is sonicated for three hours in a sonication bath (Model 2510, available from Branson). The solution is heated in a water bath at 90° C. while agitating it for ten hours. Then, the heated solution is poured gradually into distilled water, agitated thoroughly, and filtered through a membrane filter. The resultant product is dried in an oven at 80° C. for four hours to obtain ND₅-(COOH)ₙ powder.

The same process for introducing carboxyl groups to ND₅ as described above is repeated by using ND₆₀ to obtain ND₆₀-(COOH)ₙ compound.

### Example 2

The same process as described in Example 1 is repeated, except that the starting ND powder is milled before treating it with the strong acid. The ND powder may be milled by a wet milling process using zirconium beads with a size of 10-100 μm.

### Example 3

In this example, alcohol groups (OH) are introduced to the surface of ND₅. First, 100 mg of the ND₅-(COOH)ₙ compound is added to 30 mL of anhydrous tetrahydrofuran (THF), and sonication is carried out for one hour. Next, 10 mg of lithium aluminum hydride is added to the resultant THF solution, and sonication is carried out for one hour. Then, 300 mL of methanol is gradually added to the resultant solution, followed by filtration. The filtered product is dried in an oven at 80° C. for three hours to obtain powder of ND₅-(CH₂OH)ₙ compound.

### Example 4

To introduce amine groups (NH₂) to the surface of ND, 100 mg of ND₅-(CH₂OH)ₙ powder is added to 30 mL of THF, and sonication is carried out for thirty minutes. Next, 10 mg of diethyl azodicarboxylate as a coupling agent and 50 mg of phthalimide are added thereto. The resultant solution is sonicated for two hours and agitated for three hours. Then, 300 mL of methanol is poured into the resultant mixture, followed by filtration. The filtered product is dried in an oven at 80° C. for three hours. The resultant powder is introduced into 50 mL of trifluoroacetic acid (WA) and sonicated for three hours, followed by filtration. The filtered product is dried in an oven at 80° C. for three hours to obtain ND₅-(CH₂NH₂)ₙ powder. The same procedure as described above is repeated by using ND₆₀ powder to obtain ND₆₀-(CH₂NH₂) nanodiamond compound.

### Example 5

In this example, amide groups are introduced to the surface of ND₅. First, powder of the ND₅-(COOH)ₙ compound is dissolved into 50 mL of ethylenediamine. Next, 50 mg of N-[dimethylamino]-1H-1,2,3-triazo[4,5,6]pyridinylmethylene]-N-methylmethanaminium hexafluorophosphate N-oxide (HATU) is added thereto, and sonication is carried out for four hours. The reaction mixture is diluted with 200 mL of methanol, followed by filtration. The filtered product is dried in an oven at 80° C. for three hours to obtain powder of ND₅-(CONHCH₂CH₂NH2)ₙ compound.

The same procedure for introducing carboxyl groups and amide groups to ND₅ as described above is repeated by using ND₆₀ to obtain powder of ND₆₀-(CONHCH₂CH₂NH₂)ₙ compound.

### Example 6

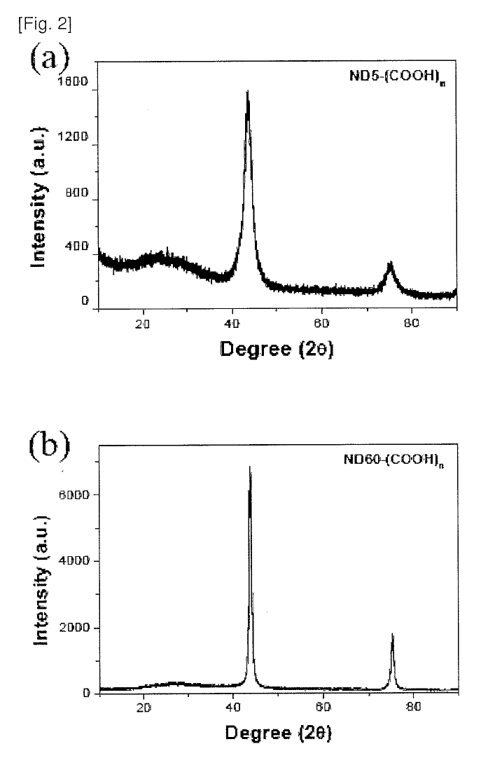

To determine the crystal structures of the powder of the ND₅-(COOH)ₙ compound and the powder of the ND₆₀-(COOH)ₙ compound, X-ray spectrum of each type of powder is obtained in a powder X-ray diffractometer (available from Rigaku) by using Ni-filtered Cu K_{α} radiation (λ=1.5418 Å). FIG. 2 shows the X-ray spectrum of each type of powder. Referring to FIG. 2, double diffraction angles (2θ) are observed at 43.84° C. and 75.21° C., which correspond to Miller indices (110) and (220) of typical diamond peaks. The average lattice constant is measured as 3.57 Å, which conforms to the reported value. This demonstrates that the above ND compounds have well-defined ND crystal structures.

### Example 7

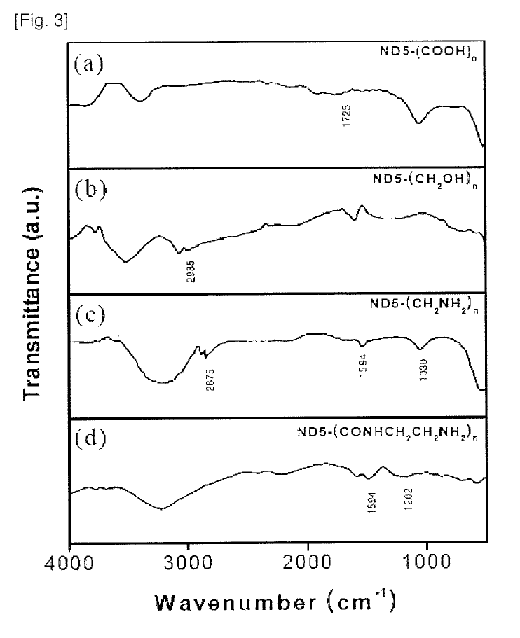

FTIR (Varian) is used to analyze the surface-modified ND compounds. The compounds are provided in the form of KBr pellets and applied to the FTIR test. FIG. 3 shows FTIR spectra of the ND₅ nanodiamond compounds.

The ND₅-(COOH)ₙ compound obtained from Example 1 shows a strong peak at a wavenumber of 1,225-1,700 cm⁻¹. This peak may be identified as a C═O stretch peak demonstrating the presence of COOH groups.

In addition, the ND₅-(CH₂OH)ₙ compound obtained from Example 4 shows no peak at 1,725-1,700 cm⁻¹ corresponding to C═O stretch, but shows peaks at a wavenumber of 2,935-2,915 cm⁻¹ and 2,865-2,845 cm⁻¹, the peaks corresponding to C—H stretching vibrations.

Further, the ND₅-(CH₂NH₂)ₙ compound obtained from Example 5 shows a peak at 1,030 cm⁻¹, which corresponds to C—N vibration. A peak corresponding to the in-plane bending mode of primary amine groups is also observed at 1,594 cm⁻¹. Additional peaks corresponding to C—H out-of-plane bending modes are observed at 700-1,000 cm⁻¹. Additionally, two peaks corresponding to stretching of CH₂ groups are observed at 2,875 cm⁻¹ and 2,895 cm⁻¹.

Finally, the IR spectrum of the ND-(CONHCH₂CH₂NH₂)ₙ compound obtained from Example 6 shows a peak corresponding to N—H bending at 1,650-1,550 cm⁻¹, and another peak corresponding to C—N bond stretching at 1,210-1,150 cm⁻¹.

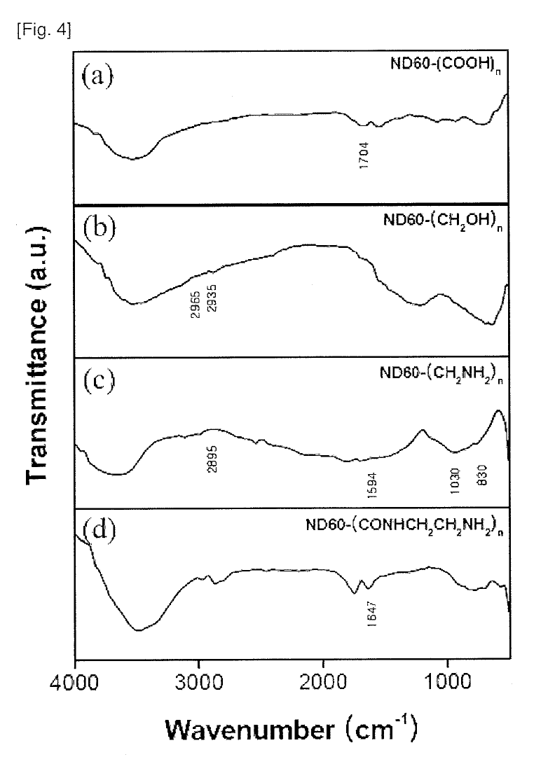

FIG. 4 shows IR spectra of the ND₆₀-Rₙ compounds. Referring to FIG. 4, it may be seen that the IR spectra of the ND₆₀-Rₙ compounds are similar to those of the ND₅-Rₙ compounds.

### Example 8

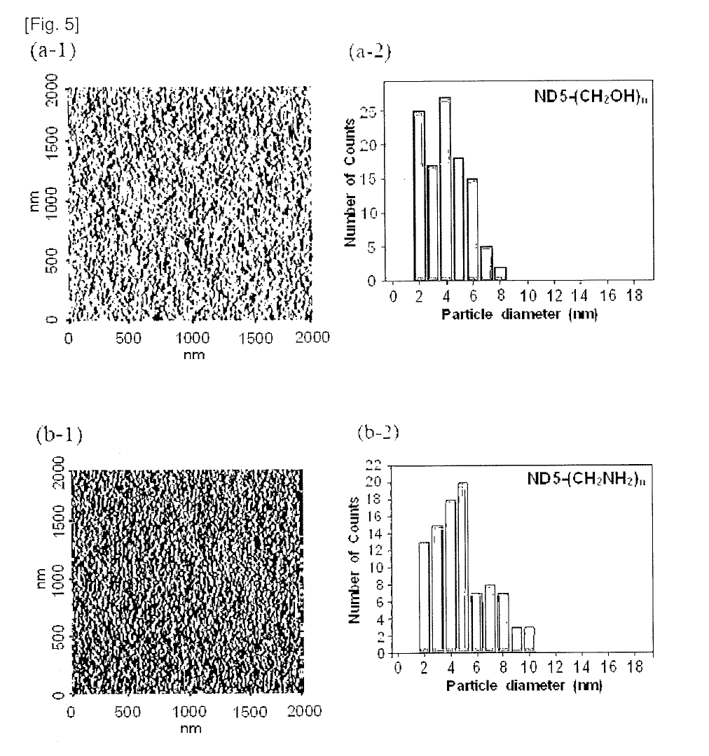

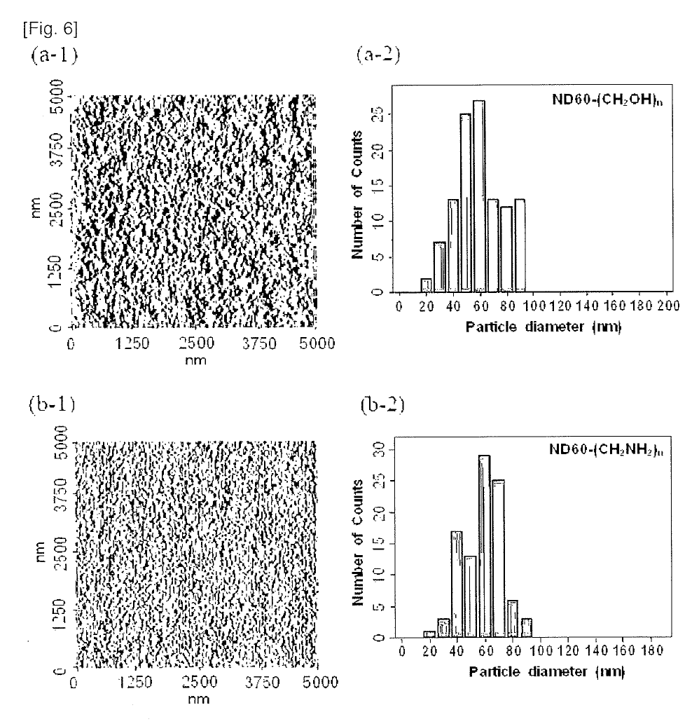

An atomic force microscope (AFM) (XE-120, available from PSIA) is used to measure the sizes of the ND₅-Rₙ compounds and ND₆₀-Rₙ compounds. First, each ND compound is dispersed in distilled water, dropped onto mica, and dried at room temperature for 24 hours. Each sample is subjected to an imaging cantilever (NCHR, available from PSIA) to obtain an image at 320 kHz in a non-contact mode under a force constant of 42 N/m. The atomic force microscope image is obtained under a pixel size of 512×512 at a scanning rate of 1 Hz. FIG. 5 shows AFM images of the ND₅-(CH₂OH)ₙ compounds and ND₅-(CH₂NH₂)ₙ compounds, as well as particle level distributions calculated therefrom. FIG. 6 shows the results of AFM for the ND₆₀-(CH₂OH)ₙ compounds and ND₆₀-(CH₂NH₂)ₙ compounds.

### Example 9

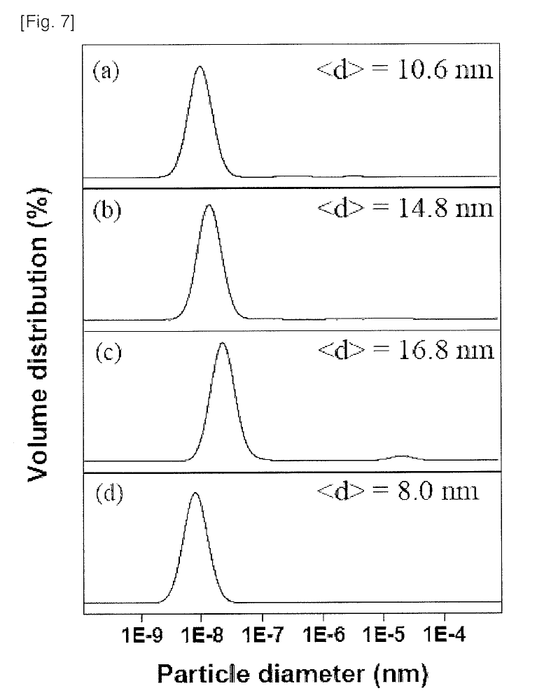

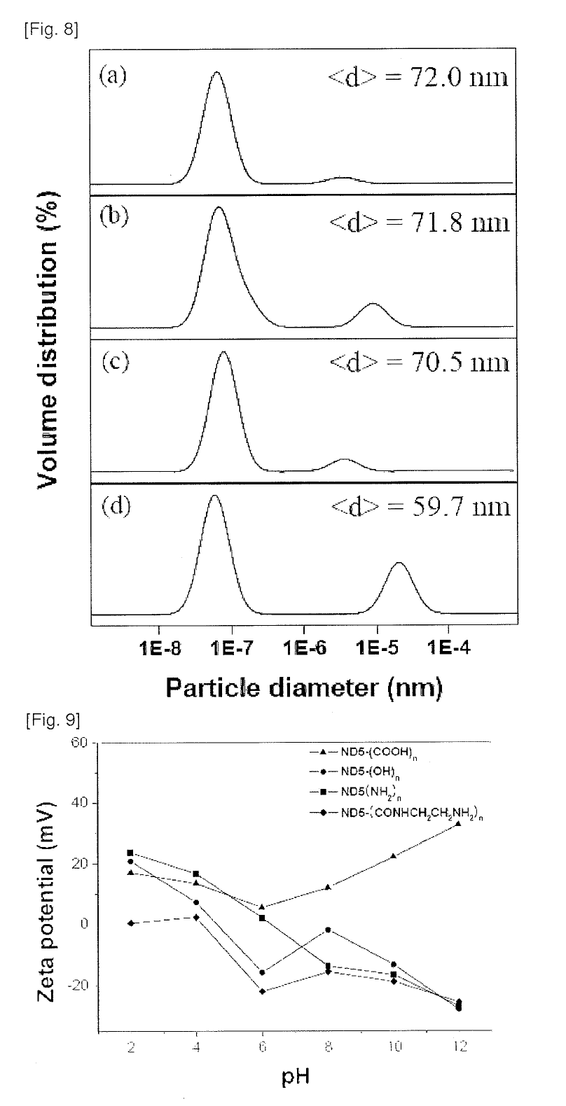

A dynamic light scattering particle size analyzer (Qudix Scateroscope I) is used to measure the particle size distributions of the ND₅-Rₙ compounds and ND₆₀-Rₙ compounds in a liquid phase. Particle size distributions in an aqueous phase (pH 7) are calculated from the autocorrelation function through reverse Laplace transformation. FIG. 7 and FIG. 8 are the results of the particle size analysis for the ND₅-Rₙ compounds and the ND₆₀-Rₙ compounds, respectively. The ND₅-Rₙ compounds are shown to have an average particle size of 8 nm-17 nm depending on the types of functional groups, while the ND₆₀-Rₙ compounds are shown to have an average particle size of 60 nm-72 nm. Among the ND₆₀-Rₙ compounds, powder of the compound functionalized with amide groups may cause partial aggregation in an aqueous phase due to the solubility of the corresponding compound. The particle size measured in a liquid phase is generally larger than the size measured by AFM. This is because the volume measured in an aqueous phase is a hydrodynamic volume. Similarly, it is thought that such variations in the particle size depending on the types of functional groups result from the interaction between the surface functional groups present on the surface of the surface-functionalized ND compound and water molecules in an aqueous phase, which leads to variations in the hydrodynamic volume of the compound.

### Example 10

To test the surface charges of the ND₅-Rₙ compounds and the ND₆₀-Rₙ compounds as a function of pH in an aqueous phase, zeta potentials of the compounds are measured by using a tester (Zetasizer, available from Malvern). First, HCl and NaOH solutions with a pH of 2, 4, 6, 8, 10 and 12 are provided, each in an amount of 1 mL. Next, 10 μL of the stock solution of each surface-functionalized ND compound is introduced to each solution, and zeta potential measurement is performed.

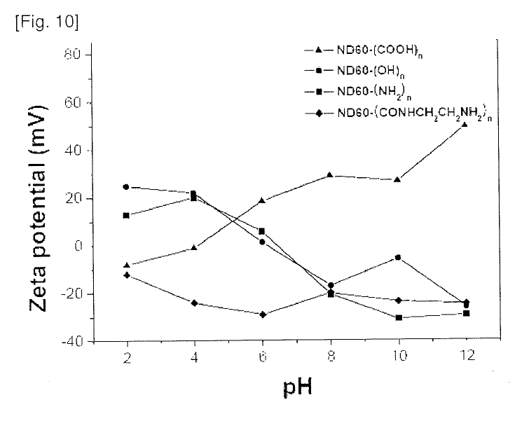

FIG. 9 is a graph showing the zeta potential measurements of the ND₅ nanodiamond compounds as a function of pH. The ND₅-(COOH)ₙ compound has a positive potential in the whole pH ranges, and is free from an isoelectric point (IEP). It is thought that the ND₅-(COOH)ₙ compound forms a stable aqueous solution in a pH range of 2-12. On the contrary, the ND₅-(CH₂OH)ₙ compound and ND₅-(CH₂NH₂)ₙ compound have an isoelectric point of 4.3 and 6.1, respectively, and the ND₅-(CONHCH₂CH₂NH₂)ₙ compound has the lowest isoelectric point of 4.0.

Referring to FIG. 9, since the ND₅-Rₙ compounds have a positive or negative zeta potential in a neutral aqueous solution, it is believed that all of the compounds are stable in a neutral pH. Referring to FIG. 10, the ND₆₀-Rₙ compounds show different behavior from the ND₅-Rₙ compounds, in a neutral aqueous solution. Particularly, the ND₆₀-(COOH)ₙ compound has an isoelectric point of 4.0, and the ND₆₀-(OH)ₙ compound and the ND₆₀-(NH₂)ₙ compound have an isoelectric point of 6.1 and 6.2, respectively. Meanwhile, the ND₆₀-(CONHCH₂CH₂NH₂)ₙ compound has no isoeletric point but has a negative zeta potential in the whole pH ranges. As a result, since the ND₅-Rₙ compounds and the ND₆₀-Rₙ compounds have a positive or negative surface charge in a neutral aqueous solution, it is believed that all of the ND compounds form a stable solution.

### Example 11

The solubility of each of the ND₅-Rₙ compounds and the ND₆₀-Rₙ compounds is measured in H₂O (pH 7), methanol, ethanol and dimethyl sulfoxide (DMSO) at 25° C. The following Table 1 shows the results of the solubility test for various surface-functionalized ND compounds.

TABLE 1

| | Solubility (g/L) | | | |
|---|---|---|---|---|
| | H₂O | | | |
| ND compounds | (pH 7) | MeOH | EtOH | DMSO |
| ND₅-Rₙ | | | | |
| ND₅-(COOH)ₙ | 140.3 | 22.7 | 10.3 | 206.3 |
| ND₅-(OH)ₙ | 156.9 | 7.1 | 3.6 | 169.1 |
| ND₅-(NH₂)ₙ | 66.1 | 9.4 | 7.1 | 135.8 |
| ND₅-(CO—NH—(CH₂)₂—NH₂)ₙ | 12.3 | 1.6 | 0.3 | 52.3 |
| ND₆₀-Rₙ | | | | |
| ND₆₀-(COOH)ₙ | 59.7 | 18.0 | 6.7 | 81.2 |
| ND₆₀-(OH)ₙ | 16.7 | 4.5 | 1.1 | 34.3 |
| ND₆₀-(NH₂)ₙ | 8.1 | 2.5 | 2.3 | 25.7 |
| ND₆₀-(CO—NH—(CH₂)₂—NH₂)ₙ | 4.2 | 0.1 | 0 | 1.8 |

Referring to Table 1, each of the compounds has the highest solubility in the polar solvent DMSO, but shows a significantly high solubility in water. Particularly, each compound may be provided as a stable solution in water, containing at most about 15% of the corresponding compound on the weight basis. Meanwhile, each compound has a relatively low solubility in an alcohol solvent as compared to DMSO and water. Particularly, the solubility in methanol is lower than the solubility in ethanol. This suggests that the solubility of each compound is related with the polarity of a solvent. The solubility test results demonstrate that the solubility increases as the particle size decreases. In general, the carboxyl-functionalized ND compound shows the highest solubility, and the solubility decreases in the order of the alcohol-, amine- and amide-functionalized ND compounds. In addition, the solubility of each compound may be increased or decreased by adjusting the pH of an aqueous solution, since the zeta potential of each compound varies with pH in an aqueous solution.

Description was made in detail with reference to example embodiments. However, it will be appreciated by those skilled in the art that changes may be made in these embodiments without departing from the principles and spirit of the method and ND compounds disclosed herein, the scope of which is defined in the accompanying claims and their equivalents.

1. A method for deaggregation and surface-carboxlyation of “hard aggregation” nanodiamond (ND) compound, which comprises the steps of: a) mixing a quantity of “hard aggregation” ND powder with strong acid solution to prepare a reaction mixture; b) sonicating the reaction mixture for more than 1 hour; c) stirring the reaction mixture between 50° C. and 100° C. for more than 3 hours; and d) putting the reaction mixture into excess pure water.

2-21. (canceled)

22. The method of claim 1, wherein the strong acid solution is selected from the group consisting of nitric acid, sulfuric acid, and combinations thereof.

23. A deaggregated and surface-carboxylated ND compound obtained by the method as defined in claim 1.

24. A method comprising the steps of: a) sonicating and then stirring a reaction mixture consisted of a “hard aggregated” ND compound and strong acid solution to provide a deaggregated and surface-carboxylated ND compound; and b) reacting the deaggregated and surface-carboxylated ND compound with a subsequent derivatizing agent along with sonication to yield a deaggregated and subsequently surface-derivatized ND compound.

25. A deaggregated and surface-derivatized ND compound obtained by the method as defined in claim 24, which has surface functionals selected from the group consisting of alkyls, esters, amines, amides, and combinations thereof.
